(19) United States
(12) Patent Application Publication
(10) Pub. No.: US 2015/0184147 A1
(43) Pub. Date: Jul. 2, 2015

(54) STRUCTURED BIOLOGICAL MATERIALS AND RELATED PRODUCTS AND METHODS

(71) Applicants: Marc Gregor Von Keitz, Minneapolis, MN (US); Jimmy Lee Gosse, Burnsville, MN (US); Stefan Thust, Minneapolis, MN (US); Erin Surdo, Minneapolis, MN (US); Jason Pratt, Pensacola, FL (US)

(72) Inventors: Marc Gregor Von Keitz, Minneapolis, MN (US); Jimmy Lee Gosse, Burnsville, MN (US); Stefan Thust, Minneapolis, MN (US); Erin Surdo, Minneapolis, MN (US); Jason Pratt, Pensacola, FL (US)

(73) Assignee: BIOCEE, INC., Minneapolis, MN (US)

(21) Appl. No.: 14/237,441

(22) PCT Filed: Aug. 3, 2012
(86) PCT No.: PCT/US12/49665

§ 371 Date: Feb. 3, 2015

Related U.S. Application Data
(60) Provisional application No. 61/515,862, filed on Aug. 6, 2011.

(51) Int. Cl. C12N 11/08; C12M 1/00; C12P 7/28; C12M 1/12; C12P 7/06; C12P 7/16

(52) U.S. Cl. CPC C12N 11/08; C12P 7/065; C12P 7/16; C12P 7/28; C12M 25/02; C12M 23/34

(57) ABSTRACT

The disclosure describes Structured Biological Materials (“SBM”) adapted for use in emulsion-less multiphasic processes. The SBM is a substratum-microbial matrix combination, wherein the microbial matrix houses one or more natural or genetically-engineered microorganisms capable of catalyzing or performing reactions that occur in multiphasic environments. When used in an emulsion-less system, the SBMs should be able to maintain separation of the bulk phases sufficient to maintain the emulsion-less nature of the system. The disclosure also provides methods of using the SBMs, for example in performance of multiphasic reactions, such as emulsion-less biphasic liquid/liquid reactions. This disclosure also provides multiphasic membrane reactors wherein an SBM functions as the membrane, such as emulsion-less biphasic reactors wherein an SBM functions as the biphasic membrane.

### CROSS-REFERENCE TO RELATED APPLICATIONS

This application claims benefit of priority to U.S. Provisional Patent Application No. 61/515,862, entitled, “STRUCTURED BIOLOGICAL MATERIALS AND RELATED PRODUCTS AND METHODS,” filed Aug. 6, 2011, which application is incorporated herein by reference in its entirety.

### STATEMENT REGARDING FEDERALLY SPONSORED RESEARCH

The government may have rights in this technology pursuant to: NSF SBIR Phase I and IB IIP-0945970; NSF/ASEE Corporate Research Postdoctoral Fellowship for Engineers Program.

### BACKGROUND

Advances in biotechnology and bioprocessing have significantly increased the opportunity to biologically transform organic chemicals with the help of whole cell biocatalysts. For hydrophobic organic chemicals, these transformations are best done in multiphasic bioprocesses, specifically liquid-liquid biphasic processes. The simplest of these biphasic processes is a direct-contact biphasic (DCB) reactor, in which the two phases (aqueous-organic) are being vigorously mixed. However, a major drawback of this reactor configuration is the formation of strong emulsions, which are further stabilized by the whole cell biocatalysts and are usually difficult to separate again. An example of one such process is the biodesulfurization (BDS) of petroleum fractions and the associated biologically catalyzed production of Hydroxy-biphenyl (HBP) and Hydroxy-biphenylsulfine (HBPS) from Dibenzothiophene (DBT). The BDS process has been scaled to several thousand liters and come close to commercialization, but ultimately was still unable to compete economically with conventional hydrodesulfurization. A detailed techno-economic analysis of BDS funded by the US Department of Energy concluded that a key problem was the presence of biologically stabilized emulsions, resulting in high cost of separating the oil and water phases and leading to significant losses of the biocatalyst. An alternative to direct-contact biphasic reactors are biphasic membrane reactors, where the aqueous phase, containing whole cell biocatalyst in suspension, is separated from the organic phase by a membrane. However, problems associated with these biphasic membrane reactors have been relatively slow mass transfer from the organic phase to the whole cell biocatalysts suspended in the aqueous phase or the breakthrough of one phase into the other across the membrane under the pressure differentials between the two phases experienced in standard reactor operating conditions.

### SUMMARY

The specification relates to Structured Biological Materials (“SBMs”) configured for use in emulsion-less multiphasic reactions. In some embodiments, an SBM is a substratum-microbial matrix combination, wherein the microbial matrix comprises at least a resin/film former and one or more microorganisms capable of catalyzing or performing reactions that occur in multiphasic environments. In some embodiments, the microbial matrix forms a layer on the substratum. In some embodiments, the substratum is incorporated into the microbial matrix layer.

In some embodiments, the microbial matrix also incorporates a porogen. In some embodiments, the microbial matrix also incorporates one or more additives, for example to provide additional functionality. In some embodiments, the microbial matrix also incorporates both a porogen and one or more optional additives. In some embodiments, the resin/film former and optional porogen(s) and additive(s) are biocompatible with the one or more microorganisms.

The specification also relates to the use of SBMs in multiphasic reactions such as emulsion-less multiphasic reactions. In some embodiment, the SBMs facilitate emulsion-less biphasic reactions, such as without limitation biological transformations involving hydrophobic compounds, such as without limitation desulfurization reactions. In other embodiments, the SBMs define a phase boundary or boundaries in emulsion-less multiphasic reactions, for example and without limitation, biphasic biological transformations involving hydrophobic compounds. In still other embodiments, the SBMs define a phase boundary or boundaries in emulsion-less multiphasic reactions, for example and without limitation the biological transformation of compounds found in crude oil or petroleum fractions such as the biologically catalyzed production of HBP. and HBPS from DBT.

The specification also relates to the emulsion-less processes for producing products in multiphasic reactions in the presence of SBMs. In some embodiments, the SBMs facilitate the processes, for example, and without limitation, catalytically. In some embodiments, the SBMs define phase boundaries in the reaction medium without the formation of an emulsion in either fluid phase. In some embodiments, the process involves contacting a first fluid phase with the SBM, and contacting a second fluid phase with the SBM. The SBM defines a boundary between the first phase and the second phase, and the microorganism(s) in the SBM transform(s) or facilitate(s) the transformation (together “transforms” from herein) of the one or more reactants into the one or more products as generalized in FIG. 1. One or more reactants can be supplied via the first fluid phase or the second fluid phase and in case of more than one reactant also via both fluid phases. Similarly, the product (or one or more of the products, if more than one product is formed) can be released into or partitioned into either the first fluid phase or the second fluid phase, or both fluid phases without the formation of an emulsion. In other embodiments, the SBMs define a phase boundary or boundaries in emulsion-less multiphasic reactions, for example and without limitation the biological transformation of compounds found in crude oil or petroleum fractions such as the biologically catalyzed production of HBP and HBPS from DBT.

The specification also relates to multiphasic membrane reactors, such as emulsion-less multiphasic membrane reactors, wherein an SBM defines the membrane. In some embodiments, the emulsion-less multiphasic membrane reactor is a biphasic membrane reactor, and an SBM defines the phase boundary. In other embodiments, the multiphasic membrane reactor is constructed for use in biological transformations involving hydrophobic compounds and an SBM defines the phase boundary. In some embodiments, the multiphasic membrane reactor is constructed for use in a BDS process, and an SBM defines the phase boundary.

The identified embodiments are exemplary only and are therefore non-limiting. The details of one or more non-limiting embodiments of the invention are set forth in the accompanying drawings and the descriptions below. Other embodiments of the invention should be apparent to those of ordinary skill in the art after consideration of the present disclosure.

### DESCRIPTION OF DRAWINGS

FIG. 1 is a diagrammatic representation of an SBM serving as a phase boundary in a biphasic medium and the biocatalyic reactions occurring with the SBM in accordance to one embodiment consistent with the disclosure.

FIG. 2 is a diagrammatic representation of an SBM serving as a phase boundary in a biodesulfurization process, and the biocatalytic reactions occurring within the SBM during the process.

FIG. 3 is a diagrammatic representation of an asymmetric SBM according to one embodiment consistent with the disclosure.

FIG. 4 is a diagrammatic representation of an asymmetric SBM according to another embodiment consistent with the disclosure.

FIG. 5 is an example of a biphasic reactor incorporating an SBM according to an embodiment consistent with the disclosure.

FIG. 6 is another example of a biphasic reactor incorporating an SBM according to another embodiment consistent with the disclosure.

FIG. 7 is another schematic of an embodiment of a biphasic reactor incorporating an SBM consistent with the disclosure.

FIG. 8 is another schematic of the biphasic reactor of FIG. 7.

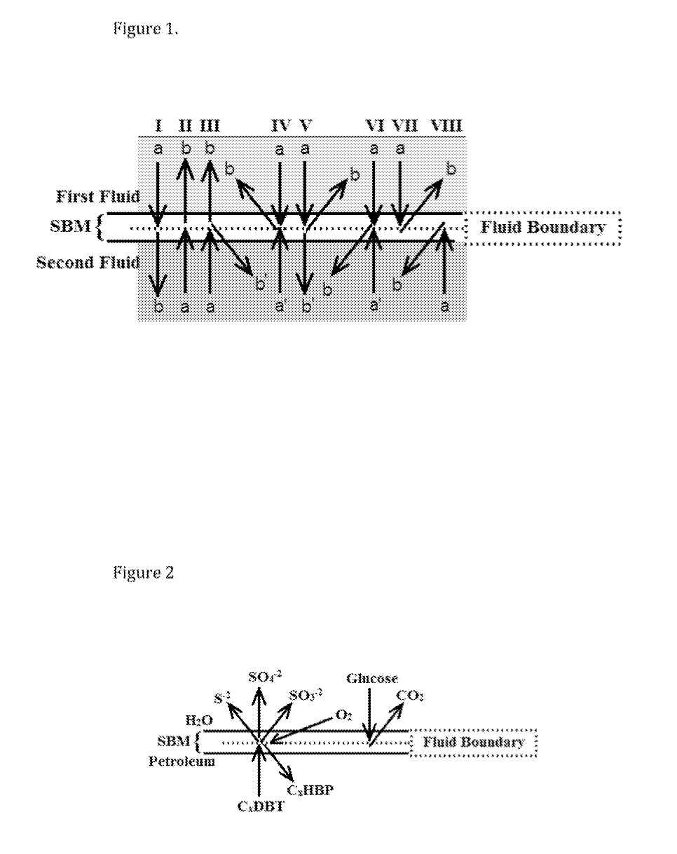

### DETAILED DESCRIPTION

### I. Definitions

Unless defined otherwise, all technical and scientific terms used herein have the same meaning as is commonly understood by one of ordinary skill in the art to which this disclosure belongs. In the event that there is a plurality of definitions for a term herein, those in this section prevail unless stated otherwise.

Where ever the phrase “for example,” “such as” and the like are used herein, the phrase “and without limitation” is understood to follow unless explicitly stated otherwise. Therefore, “for example biodesulfurization” means “for example and without limitation biodesulfurization.”

The term “about” is meant to account for variations due to experimental error. Unless explicitly stated otherwise, or nonsensical in context, all measurements or numbers are implicitly understood to be modified by the word about, even if the measurement or number is not explicitly modified by the word about.

The term “substantially” is meant to permit deviations from the descriptive term that don't negatively impact the intended purpose. Unless explicitly stated otherwise, or nonsensical in context, all descriptive terms are implicitly understood to be modified by the word substantially, even'if the descriptive term is not explicitly modified by the word substantially.

The term “substratum” when used to identify a component of the SBM means a material intended for providing mechanical support. The term “substrate” when used in connection with describing a chemical transformation means “reactant.”

The term “multiphasic” means two or more phases, for example biphasic or triphasic. The phases can be, for example, aqueous/nonaqueous liquid and aqueous liquid/gas phase.

The term “biocompatible” when used to describe the substratum and resin/film former means the materials do not as a whole negatively impact the targeted reaction catalyzed by the incorporated microorganisms such that the SBM, once made, fails to achieve the desired reactivity. In other words, the SBM fails to perform the desired reaction for a desired duration of time or fails to facilitate the production of product to a desired level. For example, in some embodiments, the SBM materials are considered “biocompatible” if the rate of reactivity after one week is no less than about 50% that of the initial reference point. As another example, in some embodiments, the rate of decline of biological activity within three months is about 20% or less than that of the reference point. In some embodiments, SBM reactivity is the rate of the desired chemical reaction to be catalyzed by the embedded microorganism, expressed per unit of surface area of the SBM (e.g. gram of product per meter square per hour) or per mass of cell (e.g. gram of product per gram of cells per hour). The “initial reference point” for defining biocompatibility is the rate of reactivity as measured after final fabrication of the SBM. In some embodiments, to account for time needed to establish a steady-state environment, the initial reference point is set as the measured reactivity 48 hours after starting the desired reaction. In some embodiments, to account for the duration of the operational lifetime, the initial reference point is set as the measured reactivity 120 hours after starting the desired reaction.

The term “genetically-engineered” when describing a microorganism means a microorganism with a genome, which has been modified, without limitation, by one or more of the following techniques: random mutagenesis, site-directed mutagenesis, directed evolution, recombinant DNA technology, or any other artificial modification of the genetic material.

The term “a” means “one or more.” For example, the phrase “the microbial matrix comprises a film former” means that the microbial matrix comprises one or more types of film formers. Similarly, the phrase “the microbial matrix houses a microorganism” means that the microbial matrix incorporates one or more types of microorganisms, such as one or more different bacteria or yeast or combinations thereof.

The phrase “configured for use in emulsion-less multiphasic reactions” when modifying SBM means that the SBM is designed and constructed to facilitate or perform a target reaction that occurs in a multiphasic system, which is “emulsion-less” as defined below. For example, the SBM is designed and constructed to facilitate or perform biodesulfurization in an emulsion-less biphasic system comprising oil and water. “DeSigned and constructed” means a target reaction, for example a biological transformation of organic compounds found in crude oil or petroleum fractures such as biodesulfurization, is chosen, a microorganism is chosen that can facilitate or perform the target reaction, a substratum, resin/film former and any optional additional materials (such as porogens and additives) are chosen, which are biocompatible with the chosen microorganism, and the SBM when made has sufficient porosity to allow the substrates of the target reaction to reach the microorganism, and the products of the target reaction to reach the appropriate phase, at a rate sufficient to sustain the desired reaction rate.

The term “phase boundary” when used to describe the SBM does not mean that the SBM is completely impermeable to one or more of the phases. Rather in some embodiments, there is penetration of the SBM by one or more of the phases such that the phase boundary is located within the SBM. Although not wishing to be bound by theory, it is believed that penetration of one or more phases is a function of the pressure differential between the two phases. The exact location of the phase boundary within the SBM may vary over time and the phases may be in an emulsion or similar state within the SBM.

The term “emulsion-less” when describing the multiphasic biological transformations involving hydrophobic compounds means that the individual fluid phases present in the multiphasic reactor are separated by the SBM and do not substantially mix outside the SBM. Although not wishing to be bound by theory, it is believed when the phase boundary is defined by the SBM and emulsion or type of emulsion may be present within the SBM and the location of the emulsion may change with time. Thus, in the multiphasic reactor outside the SBM, substantially no emulsions are created as a result of mixing of the individual fluid phases. In some embodiments, substantially free of emulsion would be considered 20% or less of the total fluid not found in the SBM would participate in the formation of an emulsion within the multiphasic reactor. In some embodiments, the amount would be 15% or less, 10% or less, 5% or less, 2% or less. And In some embodiments, the amount would be 1% or less.

An “emulsion-less” reactor is one which is capable of being used for running one or more types of emulsion-less reactions. For example, in some embodiments, an emulsion-less biphasic reactor is capable of being used for running one or more types of biphasic biological transformations, for example one or more types of biphasic liquid/liquid biological transformations. In other words, the emulsion-less reactor is not required to be able to run the full complement of emulsion-less reactions, only at least one specific emulsion-less reaction.

The term “integrated into” when describing the relationship between the substratum and microbial matrix does not mean that the substratum is necessarily completely integrated into the microbial matrix, but rather can be integrated in total or in part, as shown in FIG. 4. “Integrated in part” means that parts of the substratum are submersed in the microbial matrix. One example is the application of a microbial matrix onto the substructure side of an asymmetric porous membrane substratum with most of the substructure being embedded into the microbial matrix, while the membrane's other portion remains outside the microbial matrix.

“Microorganism(s)” is intended to be broad, encompassing the full scope of microorganisms, including natural microorganisms, genetically- or otherwise modified microorganisms unless explicitly stated otherwise.

### II. Structured Biological Materials for Multiphasic Processes

The present disclosure relates to SBMs configured for use in emulsion-less multiphasic processes. SBMs provided herein comprise a substratum and a microbial matrix, which comprises a resin/film former and one or more catalytically-active microorganisms, and optionally one or more additives, such as porogens. In some embodiments, the SBM also includes a coating on one or more of its surfaces.

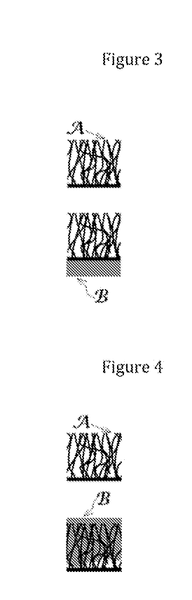

The substratum provides the microbial matrix with a surface to adhere to and also may provide additional tensile and/or compressive strength, and any manner of combining the substratum with the microbial matrix to achieve adhesion and added tensile strength is within the scope of this disclosure. For example, as shown in FIG. 3, in some embodiments the substratum and microbial matrix form a layered structure, wherein “A” is the substratum and “B” is the microbial matrix. As another example, as shown in FIG. 4, in some embodiments, the substratum (“A”) is incorporated into the microbial matrix (“B”) akin to rebar being incorporated into concrete.

The substratum can be, for example, a screen made of polymeric resin, metal, natural or synthetic fibers, the screens can be manufactured by extrusion, injection molding, weaving, or non-woven methods; a woven fabric made of synthetic or natural fibers, a non-woven fabric made of synthetic or natural fibers, paper, or a membrane.

The substratum can also offer additional functionality to the SBM, e.g. by incorporating hollow fiber membranes for gas supply or another third phase into a screen, a woven fabric, or a non-woven fabric. Another functionality that can be provided by the substratum is the delivery of light to phototrophic organisms by incorporating flexible light guides into a screen, a woven fabric, or a non-woven fabric.

In some embodiments, the substratum is permeable to one of the phases. In other embodiments, the substratum is permeable to two of the phases. In other embodiments, the substratum is permeable to each of the phases. In other embodiments, wherein the substratum is a screen, the screen has openings of about 2 mm or less. In other embodiments, wherein the substratum is a screen, and the SBM includes a coating on one or more of its surfaces, the screen openings are sized for effective coating, for example the screen openings may be 2 mm or less. In other embodiments, the substratum is stable, e.g. the substratum does not dissolve or substantially dissolve in at least one of the phases, or e.g. the substratum is not unstable under operation temperatures such as from example ranging from about 0 degrees C. to about 110 degrees C. In other embodiments, the substratum is stable to processing which occurs prior to incorporation into the SBM and is not unstable under operation temperatures such as from example ranging from 0 degrees C. to about 130 degrees C. In other embodiments, the substratum is stable to processing which occurs prior to incorporation into the SBM and is not unstable under operation temperatures such as from example ranging from 0 degrees C. to about 200 degrees C.

The microbial matrix contains microorganisms capable of facilitating or performing a desired multiphasic reaction. For example, the microorganisms can be bacteria, archaea, fungi, yeast, cyanobacteria, eukaryotic microalgae, and combinations thereof. In some embodiments, the microbial matrix comprises at least about 50% by volume cells or microorganisms.

In some embodiments, the microorganism is capable of facilitating or performing a biodesulfurization process, e.g. metabolizing an organosulfur compound as generalized for an SBM in FIG.2. In some embodiments, the microorganism capable of biodesulfurization can be Rhodococcus erythropolis, Rhodococcus rhodochrous, Pseudomonas sp, Gordonia alkanivorans, Brevibacterium sp, Paenibacillus sp, Bacillus subtilis, Myobacterium phlei, Sphingomonas sp., or any other microorganism capable of biodesulfurization as known by one of ordinary skill in the art, or mixtures thereof.

In some embodiments, the microorganism(s) will have desired hydrophobicity as measured by the bacterial adhesion to hydrocarbon (BATH) test. In some embodiments microorganisms may be chosen to have a hydrophobicity similar to Pseudomonas fluorescens. In some embodiments microorganisms may be chosen to have a hydrophobicity similar to Rhizomonas suberifaciens. In some embodiments microorganisms may be chosen to have a hydrophobicity similar to Rhodococcus erythropolis. In some embodiments microorganisms may be chosen to have a hydrophobicity similar to Acinetobacter venetianus, all as known by one of ordinary skill in the art, or mixtures thereof.

In some embodiments, the microorganism is capable of facilitating a reaction that is preferentially conducted in a biphasic reactor system, such as biocatalytic conversion with products and/or substrates that are only poorly soluble in water but well soluble in organic solvents, and biocatalytic conversions with substrates and products that are toxic to the microorganisms and that preferentially partition into an organic solvent. Examples of these types of reactions are the production of ethanol, butanol, or acetone by several species of the genus Clostridium, Zymomonas mobilis, E. coli, or various strains of yeast, such as Saccharomyces cerevisiae, the oxidation of styrene to styrene epoxide by E. coli or strains of Pseudomonas.

In some embodiments, the minimum requirement for sufficient film former/resin is chosen to achieve a stable microbial matrix, i.e. one that does not dissolve upon rehydration. The resins or film formers can be chosen from macromolecular or macromolecule-forming substances. They can be natural substances, modified natural substances or synthetic substances. In some embodiments, the resins or film formers can be formulated in a water containing solvent solution. The resins or film formers used for preparing the microbial matrix can be hydrophobic or hydrophilic. It is also possible to simultaneously use hydrophobic and hydrophilic resins or film formers (including a combination of two or more hydrophobic or hydrophilic resins or film formers) to attain bicontinuous polymer matrices. Natural substances as resins or film formers include natural resins such as shellac, oils subjected to oxidative drying such as linseed oil, tung oil, dehydrated castor oil, or fish oils, polysaccharides such as agar, agarose, pectin, or starch, or proteins such as gelatin or fibrinogen. Modified natural substances as resins or film formers include modified natural resins, modified oils, cellulose derivatives such as cellulose esters or cellulose ethers, modified natural rubber such as cyclorubber. Synthetic substances as resins or film formers include polyurethanes, polyacrylates, polyolefines, polyvinyls, synthetic rubber, cross-linked polyethylene or polypropylene glycol.

The microbial matrix can optionally include a porogen. In some embodiments, porogens are desirable to achieve sufficient porosity for the SBM to perform the desired function according to a desired efficiency. “Sufficient, porosity” is at a minimum the porosity required to transport substrates to the microorganisms and allow product to be transported out of the SBM. In some embodiments, porogens are desired to achieve the highest porosity possible without compromising the mechanical integrity and phase separation capacity of the SBM (“highest porosity”).

In some embodiments porosity (e.g. sufficient porosity or the highest porosity) can be created without porogens for example by using a high enough ratio of the microorganisms to the resin/film former.

When a porogen is used, in some embodiments, the porogen can be either dissolvable or permanent. Dissolvable porogens can include carbohydrates (e.g. glycerol, sucrose, trehalose) or gas bubbles. The gas bubbles can be the result of an entrained gas stream or they can be produced by a chemical reaction taking place during the formation of the microbial matrix, for example the release of carbon dioxide when reacting certain polyurethane components. Permanent porogens are usually filler-type materials, including particles of varying shape made from high Tg polymers or crosslinked polymers, minerals such as carbonates, silicious acids (diatomaceous earth), silicates, or glass. The permanent filler-type porogens can also be porous materials themselves, such as porous glass beads or porous chromatographic resin beads. It is possible to combine multiple porogens including dissolvable and permanent porogens.

Porosity can be measured by determining water uptake of the final material, measuring flux through the material, or via analysis of scanning electron microscopic images of the material.

The microbial matrix can also optionally include one or more additives, for example to provide additional properties or to improve properties. For example the additives can be defoaming agents, wetting and dispersing agents, surface-active additives, rheological additives (viscosity modifiers), hydrophobicity modifiers, adhesion promoters, driers and catalysts, selective biocides, pigments, solvents, fillers, light stabilizers, product adsorbents such as chromatographic resins or activated carbon, pH modifiers such calcium carbonate or ion exchange resins, electrically conductive materials such as carbon black.

In some embodiments, the substratum, such as a membrane or screen, by itself does not constitute an effective phase boundary, i.e. one or both of the phases can break through into the other phase under the operating conditions of the system. When incorporated into the SBM, the combination of the substratum and the microbial matrix is no longer permeable for one or both of the phases under the operating conditions of the system.

In some embodiments, the SBM also includes a hydrophilic coating, a hydrophobic coating or both. The hydrophilic coating can be applied to the SBM surface facing the hydrophilic phase, and the hydrophobic coating can be applied to an SBM surface facing a hydrophobic phase. The coatings generally do not themselves incorporate the microorganism and in some embodiments have pore sizes smaller than the size of the microorganisms in order to prevent or alleviate leaching of the microorganism out of the SBM.

In some embodiments, the SBM is capable of being stored dry in air or another gas at temperatures of about 273K and above for a desired time period, for example 24 hours, while still maintaining a commercially relevant level of activity without dry storage, which could be for example fifty percent of the biological activity that can be measured. In some embodiments, the SBM is capable of being stored dry for about a month, or for twenty-eight (28) days, while still maintaining commercially relevant biological activity without dry storage. In some embodiments, the SBM is capable of being stored dry for six (6) months, while still maintaining commercially relevant biological activity without dry storage. This property of the SBM enables transporting the SBM without freezing or refrigerating the SBM. A person of ordinary skill understands that “dry” does not mean complete absence of moisture, but for example “dry to the touch.”

In some embodiments, the SBM is symmetrical. In other embodiments, as shown in FIGS. 3 and 4 the SBM has directionality, i.e. the SBM is not symmetrical.

For example, in some embodiments in which the SBM is configured for use in a biphasic reaction, one side of the SBM is preferentially exposed to one phase and another side is preferentially exposed to another phase.

### III. Methods of Making Structured Biological Materials for Multiphasic Applications

To produce an SBM, cells, binder, porogens, and any additives are mixed together into a formulation, which is then applied to a substratum. The curing or solidification of the SBM is achieved by a chemical and/or physical interaction of the binder components, which can be induced by drying, cooling, or chemical reactions.

In selecting components to prepare an SBM, the starting point is the organism as it defines the desired biological functionality of the SBM. Other components are chosen to be biocompatible with the organism. Preferably, deleterious (e.g. toxic) effects of components on the organism, which reduce the desired activity of the organism, are minimized. The resin or film former is generally chosen next. The microbial matrix, once cured, should be compatible with the fluid phases comprising the reaction medium, for example with the two liquid phases in a liquid/liquid biphasic system to which the SBM will be exposed. If the microbial matrix is applied to a substratum in the form of a coating, the microbial matrix should have adhesive properties sufficient to achieve a good bond to the substratum. Some properties of different substrata are shown in Table 1. The diffusion coefficients were determined using diffusion cells and calculated using Nightingale's Equation (Cussler 1997). Also reported is η, which is the ratio of the observed diffusion coefficient through the membrane to the diffusion coefficient of the solute (DBT, nitrate) in the corresponding solvent only (hexadecane, water). F100 and F101 polypropylene membranes are available from 3M. BTS polyethersulfone and MMM 0.1 membranes are available from Pall Corporation.

TABLE 1
Summary of substratum properties

| | | Thick- | Pore | | |
|---|---|---|---|---|---|
| | | ness | size | D_{cff} | |
| Solute | Membrane | (μm) | (μm) | (cm²/s) | η |
| DBT | F100 Polypropylene | 118 | 0.2 | 1.4 × 10⁻⁶ | 0.48 |
| | F101 Polypropylene | 120 | 0.45 | 2.4 × 10⁻⁶ | 0.48 |
| | BTS Polyethersulfonc | 126 | 0.05 | 2.5 × 10⁻⁶ | 0.50 |
| Nitrate | MMM 0.1 | 143 | 0.1 | 8.6 × 10⁻⁶ | 0.45 |

In some embodiments, to be able to coat the substratum properly the hydrophobicity/hydrophilicity of the microbial matrix formulation is adjusted to achieve good wetting of the substratum. For a given substratum, the parameters that can be adjusted to tune wettability include the resin/film former, the organism, and the dosing of the specific additives.

TABLE 2
Summary of a
SBM formulation
composition. SBM's are
prepared with a formulation.

| | Base Formulation | (Weight %) | |
|---|---|---|---|
| Porogens | Wet Cell Weight | 1.2 g | (38.4) |
| | SF012 Latex | 1 ml | (56.7) |
| | Sucrose | 87.5 μl | (3.4) |
| | (0.58 g/ml) | | |
| | Glycerol | 37.5 μl | (1.5) |

(50% w/v)

In some embodiment for making an SBM for biodesulfurization, for example, a microporous polypropylene membrane is coated with a formulation containing the bacterium Rhodococcus erythropolis, Rohm & Haas SF012 latex binder emulsion, glycerol and sucrose as porogens, and no further additives. In some embodiments, the formulation contains as shown in Table 2, 1.2 g wet cell weight, 1 ml SF012 latex binder, 87.5 microliter sucrose (0.58 g/ml) and 37.5 microliters glycerol (50% w/v).

### IV. Multiphasic Processes Using Structured Biological Materials

SBMs according to the present disclosure are useful as phase boundaries in multiphasic processes, for example emulsion-less multi-phasic processes, including bi-phasic and tri-phasic applications. For example, the bi-phasic processes can be liquid/liquid processes or liquid/gas processes. The biphasic process can be any reaction which can be facilitated by a microorganism in a biphasic system. One example of such a reaction is biodesulfurization. As another example, the reactions can also be biocatalytic conversions with products and/or substrates that are only poorly soluble in water but well soluble in organic solvents, and biocatalytic conversions with substrates and products that are toxic to the microorganisms and that preferentially partition into an organic solvent. In some embodiments, the biphasic process is the transformation of organic sulfur compounds present in fossil fuel. Some examples of these types of reactions are the production of ethanol, butanol, or acetone.

### V. Multiphasic Reactors Comprising Structured Biological Materials

The specification also relates to multiphasic membrane reactors, wherein an SBM functions as the membrane (for example creates a phase boundary). In some embodiments, the SBM is deployed in emulsion-less multiphasic reactors to provide the membrane. In some embodiments, the SBM can be deployed in a two phase emulsion-less reactor system, for example wherein the SBM creates a phase boundary between the two phases. In some embodiments, the SBM maintains separation between the bulk phases in the emulsion-less system sufficient to maintain the emulsion-less nature of the system. In some embodiments, the two phase reactor system includes a basal salts media as the first fluid, and an ultra-low sulfur diesel or hexadecane spiked with dibenzothiophene as the second fluid.

FIG. 5 is a schematic diagram of one embodiment of a multiphasic reactor comprising an SBM in accordance with the present disclosure. As shown, the reactor 102 includes a reaction cell 103 which has an SBM 104 located within it. The SBM 104 is sealed to the wall of the housing 106 and divides the interior of the housing into aqueous phase chamber 108 and organic phase chamber 110. A side 105 of the SBM 104 facing the aqueous phase chamber 108 is hydrophilic. An opposing side of the SBM 104, which faces the organic phase chamber 110, is hydrophobic. The liquids in the aqueous phase chamber 108 and in the organic phase chamber 110 both penetrate into the SBM 104 and contact each other in the SBM 104. The substratum 111 is shown as a layer of the SBM 104 on the side of the SBM 104 facing the organic phase chamber 110. The substratum 111 provides mechanical support to the SBM 104.

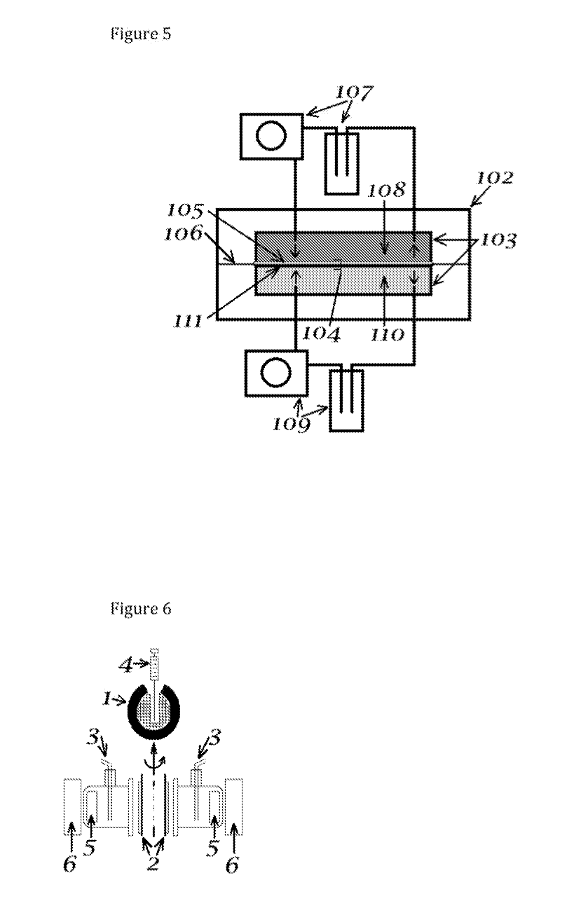

FIG. 6 is a schematic diagram of another embodiment of an emulsion-less multiphasic reactor comprising an SBM in accordance with the present disclosure. More specifically, FIG. 6 is a schematic diagram of a Micro-BDS reactor configuration in accordance with an embodiment consistent with the present disclosure. As shown, the reactor holds about 9.8 cm² of SBM in an emulsion-less biphasic reactor configuration. Each glass half-cell contains aqueous media. A Teflon ring and screen spacer (together 1) is sandwiched between two SBM 2 cut in the shape of round discs creating a space for loading and sampling 4 the organic phase. The SBM 2 comprises a microbial matrix facing the aqueous phase on a microporous polypropylene membrane (3M, F100) substratum facing the organic phase. Also shown are aeration lines 3, which are contained in each half-cell, magnetic stir bars 5 and magnetic stir plates 6. The MicroBDS reactor and its use are further described in Example A, B and C, below.

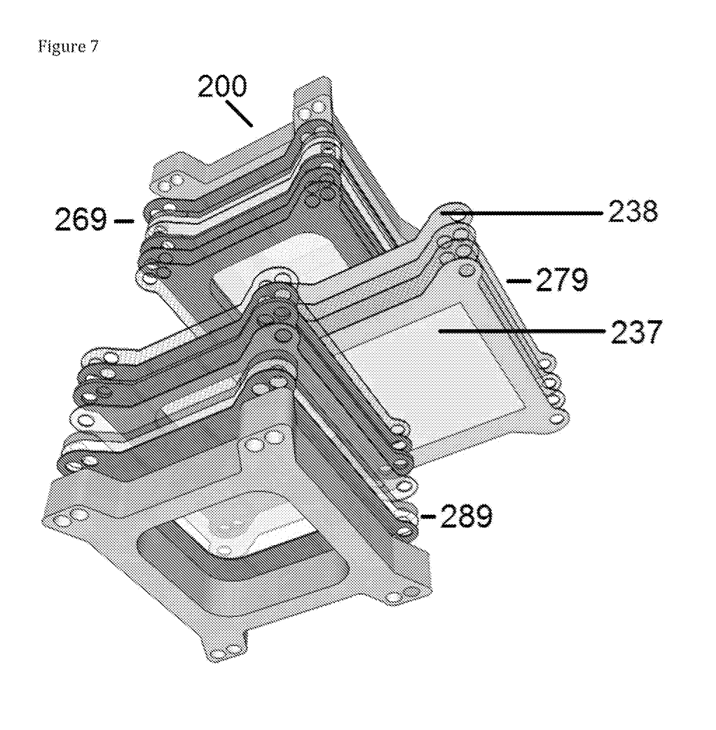

FIG. 7 is a schematic diagram of another embodiment of an emulsion-less multiphasic reactor comprising an SBM in accordance with the present disclosure. More specifically, FIG. 7 is a schematic diagram of a biphasic emulsion-less BDS reactor configuration in accordance with an embodiment consistent with the present disclosure. As shown, the reactor holds about 150 cm² of SBM 237, 238 in an emulsion-less biphasic reactor configuration. The reactor 200 can be divided into the sections representing phase 1269, phase II 279 and phase I′ 289. Phase I 269 and phase I′ 289 contain one of the phases and phase II 279 contains the second phase. More specifically in the biphasic emulsion-less reactor configuration for BDS, phase 1269 and phase I′ 289 contain aqueous medium and phase II 279 contains the organic phase. Even more specifically, phase II 279 main contain without limitation crude oil, petroleum fractions such as high sulfur middle distillate, ultra-low sulfur diesel, hexadecane or combinations thereof. The reactor and its use are further described in Example D, below.

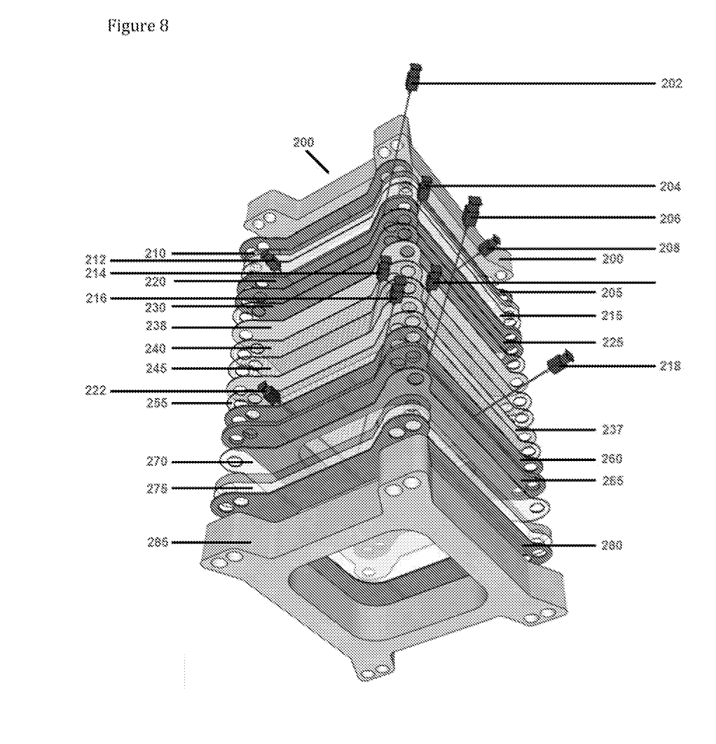

FIG. 8 is another schematic diagram of the emulsion-less multiphasic reactor 200 comprising an SBM shown in FIG. 7 in accordance with the present disclosure. The reactor is assembled with aluminum housing 201, 285, viton gaskets 205, 220, 225, 260, 265, 280, 6 mm acrylic plates 210, 275, polyester sheets 215, 270, polyester screen gasket 230, 240, 245, 255 and SBMs 237 and 238. The reactor is assembled with stainless steel luer lock needles for adding and removing the contents of the fluid phases as well as air. The stainless steel luer lock needle 202 is air out of phase I, luer lock needle 204 is air inlet to phase I, luer lock needle 206 is air out phase I′, luer lock needle 208 is fluid out phase I, luer lock needle 212 is fluid in phase I, luer lock needle 214 is fluid out phase II, luer lock needle 216 is air in phase I′, luer lock needle 218 is fluid out phase I′, luer lock needle 222 is fluid in phase I′. Not shown in FIGS. 6 and 7 are the fasteners, hoses, connectors, pumps, reservoirs, and stand as can be appreciated and understood by one of ordinary skill in the art upon reading this disclosure.

### Examples

A. Preparing a Structured Biological Material for screening of biodesulfurization activity using an organic phase containing a single or multiple sulfur sources such as dibenzothiophene (DBT) in a biphasic emulsion-less micro-biodesulfurization (microBDS) reactor.

### Microorganism.

Rhodococcus erythropolis (R. erythropolis) was prepared by harvesting a cell culture at an OD between 1.0 and 1.5. The culture was centrifuged at 5000 rpm and 4° C. for 10 minutes. The initial cell pellet was resuspended in BSM2 medium (about 45 ml per 400 ml original culture) and re-centrifuged at the same conditions for 10 minutes in a 50 ml conical tube. The pellet was weighed and recorded as the wet cell weight (WCW)

### Formulation.

The coating formulation was prepared by adding sucrose (580 g/L, Fisher

Scientific) and glycerol (500 g/L, Fisher Scientific) to the wet cell pellet according to the following; 1.2 g WCW, 1 ml Rhoplex SF-012 latex (Rohm and Haas, DOW), 87.5 μl sucrose and 37.5 μl glycerol. Cells were gently mixed with the sucrose and glycerol solutions before the latex was added. After latex addition the formulation was mixed.

### Substratum.

Polypropylene membranes (F100, 3M) were masked with one layer of vinyl adhesive (Con-Tact) around the edges to create a defined coating surface area of about 6×10 cm on the membrane with a mask depth of about 85 μm.

### Creating the Structured Biological Material.

To create the SBM the formulation was drawn down with a #28 Mayer Rod on the substratum and allowed to dry for 60 min in a controlled environmental chamber set at 25° C. and 50% relative humidity.

### Biodesulfurization in a Biphasic Emulsion-Less MicroBDS Reactor.

A microBDS R. erythropolis SBM emulsion-less biphasic reactor was assembled as shown in FIG. 6 with the organic phase contacted on both sides by the F100 substratum component of the SBMs. The microbial matrix component of each SBM faces the aerated aqueous phase in each half cell for a total SBM surface area of 9.8 cm². The biphasic reactor was charged each day with BSM2 medium and hexadecane containing DBT at a concentration of 103 ppm S. At the end of each day (7 hours), the reactor was recharged by refilling with fresh hexadecane containing DBT at a concentration of 200 ppm S (data not shown).

Dataset 1, below, shows the results of a biodesulfurization experiment completed in the microBDS reactor.

### B. Dry Storage of SBM

Three SBMs were prepared as described in Example A. After preparing the SBMs, two were removed from the glass support and placed into storage bags, sealed and stored in the dark at room temperature. After a predetermined

TABLE 3
SBM BDS rates after
increasing durations of
storage. Storage does not
significantly diminish BDS rate.

| | BDS rate |
|---|---|
| Storage Time | (ppm S h−1) |
| None | 4.04 |
| One week | 4.73 |
| Four weeks | 3.58 |

storage time (one week and four weeks) the SBMs were removed and then loaded into the microBDS reactor as previously described in Example B. One of the SBMs was loaded without storage and the reactivity monitored immediately. Table 3 shows the rates of biodesulfurization observed for each case.

### C. Preparing a Structured Biological Material of Differing Composition.

TABLE 4
MicroBDS rates observed for
SBM's prepared with Rhoplex SF012
and various polyurethane matrices.
The prepared SBM's can be screened for
activity. Error ± STD of n = 9.

| | DBT Degradation Rate |
|---|---|
| | (ppm S/h) |
| SF012 Control | 7.3 ± 1.5 |
| Baycusan C1000 | 7.4 |
| Baycusan C1000 | 10.8 |
| Baycusan C1001 | 9.6 |
| Baycusan C1003 | 7.6 |
| Baycusan C1004 | Unstable |
| Impranil DLU | 6.8 |

SBM's were prepared as described in Example A with the exception that the polymeric matrices shown in Table 4 were used in place of SF012. The SBM's were then screened for activity in a method similar to Example B. The polymeric matrices Baycusan C1000, Baycusan C1001, Baycusan C1003, Baycusan C1004 and Impranil DLU are polyurethane dispersions available from Bayer MaterialScience.

Additional SBM's were prepared as described in Example A with the exception that differing amounts of wet cell pellet and porogens were used in the matrices shown in Table 5. The SBM's were then screened for activity in a method similar to Example B.

TABLE 5
MicroBDS rates observed for
SBM's prepared with Rhoplex SF012 and
Baycusan C1000 with various cell and
porogen loadings. The composition of the
formulation used to create the SBM can be
varied.

| | DBT Degradation |
|---|---|
| | rate (ppm S/h) |
| SF012 Control | 7.3 ± 1.5 |
| SF012 1.5xBiomass | 7.7 |
| Baycusan C1000 | 9.2 |
| 3xBiomass + 3xPorogcn | |
| Baycusan C1000 3xPorogen | Unstable |

Additional SBM's were prepared as described in Example A with the exception that an additional porogen (diatomaceous earth) was added as shown in Table 6. The SBM's were then evaluated to determine diffusion coefficients diffusion cells and calculated using Nightingale's Equation (Cussler 1997). Also reported is η, which is the ratio of the observed diffusion coefficient to the

TABLE 6
Characterization of SBM's prepared with
Rhoplex SF012 and diatomaceous earth. The composition
of the formulation used to create the SBM can be varied.

| Solute | Formulation | Thickness | D_{cff} (cm²/s) | η |
|---|---|---|---|---|
| DBT | Control | 37 | 6.3 × 10⁻⁸ | 0.0126 |
| | 1 wt % DE | 69 | 1.8 × 10⁻⁷ | 0.0359 |
| | 4 wt % DE | 23 | 7.9 × 10⁻⁸ | 0.0158 |
| Nitrate | Control | 46 | 3.9 × 10⁻⁹ | 0.0002 |
| | 1 wt % DE | 59 | 1.6 × 10⁻⁷ | 0.0086 |
| | 4 wt % DE | 44 | 4.1 × 10⁻⁸ | 0.0022 |

diffusion coefficient of the solute (DBT, nitrate) in the corresponding solvent (hexadecane, water).
D. Preparing a Structured Biological Material Biodesulfurization of an Organic Phase Containing a Single or Multiple Sulfur Sources such as Dibenzothiophene (DBT) in a Biphasic Emulsion-Less Membrane Reactor.

SBM's were prepared as described in Example A with the exception that the substratum and vinyl adhesive were larger. The R. erythropolis SBM emulsion-less biphasic reactor was assembled as shown in FIGS. 7 and 8 with the organic phase contacted on both sides by the F100 substratum component of the SBMs. The microbial matrix component of each SBM faces the aerated aqueous phase in each half cell for a total SBM surface area of about 150 cm².

The results shown in dataset 2 are from a biphasic emulsion-less membrane reactor charged with BSM2 medium and hexadecane containing DBT at a concentration of about 225 ppm S. DBT removal from the emulsion-less organic phase was measured by GC-MS (DBT analysis) corresponds with sulfur reduction as determined by the ASTM D5453 method (S analysis).

SBM's were prepared as described in Example A with the exception that the substratum and vinyl adhesive were larger. The R. erythropolis SBM emulsion-less biphasic reactor was assembled as shown in FIGS. 7 and 8 with the organic phase contacted on both sides by the F100 substratum component of the SBMs. The microbial matrix component of each SBM faces the aerated aqueous phase in each half cell for a total SBM surface area of about 150 cm².

The results shown in dataset 3 are from a biphasic emulsion-less membrane reactor charged with BSM2 medium and 10% high sulfur middle distillate diluted with ultra-low sulfur diesel (ULSD) to have a sulfur concentration of about 1,250 ppm S. DBT removal from the emulsion-less organic phase was measured by ASTM D5453.

A number of embodiments have been described. Nevertheless it will be understood that various modifications may be made without departing from the spirit and scope of the invention. Accordingly, other embodiments are included as part of the invention and may be encompassed by the attached claims. Furthermore, the foregoing description of various embodiments does not necessarily imply exclusion. For example, “some” embodiments or “other” embodiments may include all or part of “some”, “other” and “further” embodiments within the scope of this invention.

For example, the embodiments include:

1. A Structured Biological Material, comprising:
a. a microbial matrix comprising: a resin, a film former, or combinations thereof, and a catalytically-active microorganism; and,
b. a substratum;
   wherein the Structured Biological Material is configured for use in an emulsion-less multiphasic system.

2. A Structured Biological Material according to embodiment 1, wherein the multiphasic system is a liquid/liquid (such as aqueous liquid/nonaqueous liquid) biphasic system.

3. A Structured Biological Material according to embodiments 1 or 2, wherein the microorganism maintains catalytic-activity for a desired amount of time in the Structured Biological Material.

4. A Structured Biological material according to embodiment 3, wherein the desired amount of time is a commercially-relevant amount of time.

5. A Structured Biological Material according to any of embodiments 1-4, wherein the emulsion-less biphasic system comprises a first bulk liquid phase and a second bulk liquid phase and the Structured Biological Material, when in use in an emulsion-less biphasic system, is able to maintain separation between the first and second bulk phase sufficiently to maintain the biphasic system as an emulsion-less system.

6. A Structured Biological Material according to any of embodiments 1-5, wherein the microbial matrix further comprises a porogen and optionally comprises an additive.

7. A Structured Biological Material according to any of the preceding embodiments, wherein the Structured Biological Material is adapted to define a phase boundary in biphasic applications.

8. A Structured Biological Material according to any of the preceding embodiments, wherein the microbial matrix has sufficient porosity to allow the reactants and products of the target reaction to reach the microorganism at rate sufficient to sustain the desired reaction rate.

9. A Structured Biological Material according to any of the preceding embodiments, wherein the substratum provides tensile strength to the Structured Biological material.

10. A Structured Biological Material according to any of the preceding embodiments, wherein the substratum forms a first layer and the microbial matrix forms a second layer proximal the first layer.

11. A Structured Biological Material according to any of the preceding embodiments, wherein the substratum is integrated into the microbial matrix.

12. A Structured Biological Material according to embodiment 4, wherein the at least one porogen is chosen from carbohydrates, diatomaceous earth, gas bubbles, and combinations thereof.

13. A Structured Biological Material according to embodiment 4, wherein the at least one porogen is chosen from glycerol, sucrose, trehalose, and combinations thereof.

14. A Structured Biological Material according to any of the preceding embodiments, wherein the substratum is chosen from a screen made of polymeric resin, metal, natural or synthetic fibers; a woven fabric made of synthetic or natural fibers; and, a non-woven fabric made of synthetic or natural fibers, paper, or a membrane.

15. A Structured Biological Material according to embodiment 4, wherein the at least one additive is chosen from viscosity modifiers, hydrophobicity modifiers, adsorbents, pH modifiers, colorants, and electrically conductive materials.

16. A Structured Biological Material according to any of the preceding embodiments, wherein the resin/film former is chosen from natural, modified natural and synthetic macromolecular or macromolecule-forming substances.

17. A Structured Biological Material according to embodiment 16, wherein the natural substances are chosen shellac, oils subjected to oxidative drying such as linseed oil, lung oil, dehydrated castor oil, and fish oils, polysaccharides such as agar, agarose, pectin, and starch, and proteins such as gelatin or fibrinogen, the modified natural substances are chosen from modified natural resins, modified oils, cellulose derivatives such as cellulose esters and cellulose ethers, and modified natural rubber such as cyclorubber, and wherein the synthetic substances as resins are chosen from polyurethanes, polyacrylates, polyolefines, polyvinyls, synthetic rubber, cross linked polyethylene and polypropylene glycol.

18. A Structured Biological Material according to any of the preceding embodiments, wherein a hydrophilic or hydrophobic coating without cells can be applied to one surface of the material.

19. A Structured Biological Material according to any of the preceding embodiments, wherein the Structured Biological Material can be stored “dry” in air or another gas at temperatures above 273 K for at least 24 hours and still maintain at least half of the biological activity that can be measured without dry storage.

20. A Structured Biological Material according to embodiment 19, wherein the Structured Biological Material can be stored dry in air for up to six months.

21. A Structured Biological Material according to any of the preceding embodiments, wherein the at least one catalytically-active microorganism is chosen from bacteria, archaea, fungi, yeast, cyanobacteria and eukaryotic microalgae.

22. A Structured Biological Material according to embodiment 21, wherein the catalytically-active microorganism is chosen from Rhodococcus erythropolis, Rhodococcus rhodochrous, Pseudomonas sp, Gordonia alkinovorans, Brevibacterium sp, Paenibacillus sp, Bacillus subtilis, Myobacterium phlei, Sphingomonas sp., and combinations thereof.

23. A Structured Biological Material according to embodiment 21, wherein the catalytically-active microorganism has a hydrophobicity as measured by the bacterial adhesion to hydrocarbon test similar to one of Pseudomonas fluorescens, Rhizomonas suberifaciens, Rhodococcus erythropolis or Acinetobacter venetianus.

24. A Structured Biological Material according to any of the preceding embodiments, wherein the microbial matrix comprises at least 50% by volume microorganisms.

25. A process for producing a product in a biphasic medium, comprising: contacting a first phase with a Structured Biological Material, and contacting a second phase with the Structured Biological Material, wherein one or more substrates are present in the first phase, the Structured Biological Material forms a boundary between the first phase and the second phase, and microorganism in the Structured Biological Material chemically transforms the one or more substrates and produce reactants into the second phase.

26. A process according to embodiment 25, wherein the Structured Biological Material is a Structured Biological Material according to any one of embodiments 1-24.

27. A process for producing a product in a biphasic system according to embodiments 25 or 26, wherein the biphasic system is an emulsion-less biphasic system and the Structured Biological Material is capable of maintaining separation between the two liquid phases sufficient to maintain the biphasic system as an emulsion-less system.

28. A process according to embodiments 25, 26, or 27 wherein the process is the transformation of organic sulfur compounds present in fossil fuel, the first phase is an organic oil phase, and the second phase is an aqueous phase.

29. A multiphasic membrane reactor, comprising:
a. a housing defining at least a first and second chamber for containing a first and second phase respectively; and,
b. a Structured Biological Material defining a membrane between the at least first and second phase, wherein the Structure Biological Material is configured to maintain separation between the at least first and second phase sufficient to maintain an emulsion-less system, and the Structured Biological Material is according to any of claims 1-24.

30. A multiphasic membrane reactor according to embodiment 29, wherein the reactor is a liquid/liquid biphasic reactor.

1. A Structured Biological Material, comprising
a. a microbial matrix comprising:
i. a resin, a film former, or combinations thereof; and,
ii. a catalytically-active microorganism; and,
b. a substratum;
wherein the Structured Biological Material is configured for use in an emulsion-less multiphasic system.

2. A Structured Biological Material according to claim 1, wherein the microbial matrix further comprises a porogen and optionally comprises an additive.

3. A Structured Biological Material according to claim 1, wherein the microorganism is capable of maintaining catalytic-activity for a desired amount of time in the Structured Biological Material.

4. A Structured Biological Material according to claim 1, wherein the emulsion-less multiphasic system comprises a first bulk phase and a second bulk phase and the Structured Biological Material, when in use in the emulsion-less multiphasic system, is able to maintain separation between the first and the second bulk phase to a degree that maintains the multiphasic system as an emulsion-less system.

5. A Structured Biological Material according to claim 1, wherein the catalytically-active microorganism is chosen from bacteria, archaea, fungi, yeast, cyanobacteria and eukaryotic microalgae.

6. A Structured Biological Material according to claim 1, wherein the catalytically-active microorganism is chosen from Rhodococcus erythropolis, Rhodococcus rhodochrous, Pseudomonas sp, Gordonia alkanivorans, Brevibacterium sp, Paenibacillus sp, Bacillus subtilis, Myobacterium phlei, Sphingomonas sp., and combinations thereof.

7. A Structured Biological Material according to claim 1, wherein the catalytically-active microorganism has a hydrophobicity as measured by the bacterial adhesion to hydrocarbon test similar to one of Pseudomonas fluorescens, Rhizomonas suberifaciens, Rhodococcus erythropolis or Acinetobacter venetianus.

8. A Structured Biological Material according to claim 1, wherein the microbial matrix comprises at least 50% by volume microorganisms.

9. A Structured Biological Material according to claim 1, wherein the Structured Biological Material can be stored dry in air at temperatures of about 273K and above for at least 24 hours or more while still maintaining a commercially-relevant level of activity.

10. A Structured Biological Material according to claim 9, wherein the Structured Biological Material can be stored dry in air at temperatures of about 273K and above for up to six months while still maintaining a commercially-relevant level of activity.

11. A process for producing a product in an emulsion-less biphasic liquid/liquid system, comprising: contacting a first phase with a Structured Biological Material, and contacting a second phase with the Structured Biological Material, wherein one or more substrates are present in the first and second phase, the Structured Biological Material forms a boundary between the first phase and the second phase, and microorganism in the Structured Biological Material chemically transforms the one or more substrates and produce reactants into the first and or second phase.

12. A process according to claim 11, wherein the process is a transformation of organic sulfur compounds present in fossil fuel, the first phase is an organic oil phase, and the second phase is an aqueous phase.

13. A multiphasic membrane reactor, comprising:
a. a housing defining at least a first and a second chamber for containing a first and a second phase respectively; and,
b. a Structured Biological Material defining a membrane between the at least first and second phase, wherein the Structured Biological Material is configured to maintain separation between the at least first and second phase sufficient to maintain an emulsion-less system, and the Structured Biological Material comprises:
i. a microbial matrix comprising a film former, a resin, or combinations thereof, and a catalytically-active microorganism; and,
ii. a substratum.

14. A reactor according to claim 13, wherein the microorganism maintains catalytic activity for a desired amount of time in the Structured Biological Material and in the emulsion-less multiphasic system.

15. A reactor according to claim 13, wherein the reactor is a liquid/liquid biphasic membrane reactor.

16. A Structured Biological Material according to claim 1, wherein the substratum provides tensile strength to the Structured Biological material.

17. A Structured Biological Material according to claim 1, wherein the substratum forms a first layer and the microbial matrix forms a second layer proximal the first layer.

18. A Structured Biological Material according to claim 1, wherein the substratum is integrated into the microbial matrix.

19. A Structured Biological Material according to claim 1, wherein the substratum is chosen from a screen made of polymeric resin, metal, natural or synthetic fibers, a woven fabric made of synthetic or natural fibers, a non-woven fabric made of synthetic or natural fibers, paper, and a membrane.
